(19) United States
(12) Patent Application Publication
(10) Pub. No.: US 2024/0398556 A1
(43) Pub. Date: Dec. 5, 2024

(54) LOW PROFILE FINAL SEAL FOR HEART VALVE PROSTHESIS

(71) Applicant: MEDTRONIC, INC., Minneapolis, MN (US)

(72) Inventors: Yogesh A. Darekar, Irvine, CA (US); Tiffany T. Dang, Garden Grove, CA (US)

(21) Appl. No.: 18/697,472

(22) PCT Filed: Sep. 28, 2022
(86) PCT No.: PCT/IB2022/059260

§ 371 Date: Mar. 31, 2024

Related U.S. Application Data
(60) Provisional application No. 63/251,338, filed on Oct. 1, 2021.

(51) Int. Cl. A61F 2/24

(52) U.S. Cl. CPC A61F 2/2418; A61F 2/2415; A61F 2220/0075; A61F 2240/001; A61F 2250/0069

(57) ABSTRACT

A transcatheter heart valve prosthesis including a frame, an inner skirt, an outer skirt, and a skirt seal. The frame includes an inner portion configured to support the prosthetic valve component. The outer portion being sized to surround the inner portion and configured to anchor the prosthesis. The inner skirt has a first edge and is disposed within and coupled to the inner portion. The outer skirt has a first edge and is disposed within and coupled to the outer portion. The skirt seal is configured to provide a seal between the first edges, the skirt seal includes a first set of stitches configured to align the edges and forming a seam disposed inwardly of the first edges. The skirt seal includes a set of second stitches configured to couple the first edges, the set of second stitches being formed at the first edges to strengthen the seal.

### FIELD

The present technology is generally related to heart valve prostheses implantable via minimally invasive procedures, and in particular is directed to mitral valve prostheses having a low profile.

### BACKGROUND

The human heart is a four chambered, muscular organ that provides blood circulation through the body during a cardiac cycle. The four main chambers include the right atrium and right ventricle which supplies the pulmonary circulation, and the left atrium and left ventricle which supplies oxygenated blood received from the lungs into systemic circulation. To ensure that blood flows in one direction through the heart, atrioventricular valves (tricuspid and mitral valves) are present between the junctions of the atrium and the ventricles, and semi-lunar valves (pulmonary valve and aortic valve) govern the exits of the ventricles leading to the lungs and the rest of the body. These valves contain leaflets or cusps that open and shut in response to blood pressure changes caused by the contraction and relaxation of the heart chambers. The valve leaflets move apart from each other to open and allow blood to flow downstream of the valve, and coapt to close and prevent backflow or regurgitation in an upstream manner.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems, and are delivered in a radially compressed or crimped configuration for advancement through the patient's vasculature. Accordingly, once positioned at a treatment site, a heart valve prosthesis may be expanded, or permitted to return to an uncompressed state, to engage tissue at the diseased heart valve region to, for instance, hold the heart valve prosthesis in position.

While these valve prostheses offer minimally invasive methods for heart valve repair and/or replacement, challenges remain such as reducing a profile of a heart valve prosthesis while maintaining required performance in vivo. One challenge that relates to providing a mitral valve prostheses with a lower profile is minimizing the unintended movement of blood between the atrium and the ventricle, otherwise known as regurgitation. Current solutions use a combination of skirts positioned about a heart valve prosthesis. However, many current skirt configurations may be too thick/large so as to not permit a suitable reduction in profile for a mitral valve prosthesis. Therefore, there exists a need for improved skirt configurations in order to reduce an overall profile size of a mitral valve prosthesis while maintaining proper sealing thereabout when implanted at a target location.

The present disclosure relates to improvements in a heart valve prosthesis to ensure that the heart valve prosthesis has a low profile for transcatheter delivery through a patient's vasculature and to address proper sealing thereof when implanted.

### SUMMARY

According to a first embodiment hereof, the present disclosure provides a transcatheter heart valve prosthesis which includes a frame including an inner portion configured to support a prosthetic valve component and an outer portion coupled to the inner portion, the outer portion being sized to surround the inner portion and configured to anchor the prosthesis. An inner skirt disposed within and coupled to the inner portion, the inner skirt having a first edge. An outer skirt disposed within and coupled to the outer portion, the outer skirt having a first edge. A skirt seal configured to provide a seal between the first edges of the inner skirt and the outer skirt. The skirt seal including a set of first stitches configured to align the first edge of the inner skirt with the first edge of the outer skirt, the set of first stitches forming a seam that is disposed/spaced inwardly of the first edges of the inner and outer skirts. A set of second stitches configured to couple the first edge of the inner skirt with the first edge of the outer skirt, the set of second stitches being formed at the first edges of the inner and outer skirts. Wherein the first stitches are a first type of stitch and the second stitches are a second type of stitch different from the first type of stitch.

In an aspect of the first embodiment, and in combination with any other aspects herein, the disclosure provides that the inner portion has a stent-like structure with a first end and a second end, the stent-like structure including a plurality of crowns defining apertures at the first end.

In an aspect of the first embodiment, and in combination with any other aspects herein, the disclosure provides that the outer portion has a stent-like structure with a first end and a second end, the stent-like structure including a plurality of crowns defining apertures at the first end.

In an aspect of the first embodiment, and in combination with any other aspects herein, the disclosure provides that the plurality of the crowns at the first end of the outer portion are coupled to the plurality of crowns at the first end of the inner portion so as to form a plurality of pairs of adjoining crowns.

In an aspect of the first embodiment, and in combination with any other aspects herein, the disclosure provides that the first edge of the inner skirt is an outflow edge and the first edge of the outer skirt is an outflow edge.

In an aspect of the first embodiment, and in combination with any other aspects herein, the disclosure provides that the same number of second stitches are disposed within each pair of adjoining crowns.

In an aspect of the first embodiment, and in combination with any other aspects herein, the disclosure provides exactly three second stitches are disposed within each pair of adjoining crowns.

In an aspect of the first embodiment, and in combination with any other aspects herein, the disclosure provides that the first type of stitch is a double running stitch.

In an aspect of the first embodiment, and in combination with any other aspects herein, the disclosure provides that the first type of stitch is a single running stitch.

In an aspect of the first embodiment, and in combination with any other aspects herein, the disclosure provides that the second type of stitch is one of a single whipstitch or a double whipstitch.

In an aspect of the first embodiment, and in combination with any other aspects herein, the disclosure provides that the set of first stitches and the set of second stitches are formed by a suture having a diameter of between 50 μm and 69 μm.

According to a second embodiment hereof, and in combination with any other aspects herein, the disclosure provides a transcatheter heart valve prosthesis including a frame having an inner portion having a plurality of crowns at an outflow end thereof and an on outer portion having a plurality of crowns at a first end thereof, the plurality of crowns of the outer portion being coupled to the plurality of crowns of the inner portion so as to form a plurality of pairs of adjoining crowns. A prosthetic heart valve coupled to the inner portion. An inner skirt coupled to the inner portion, the inner skirt having a first edge. An outer skirt coupled to the outer portion and having a first edge. A skirt seal configured to provide a seal between the first edges of the inner skirt and the outer skirt. The skirt seal including a set of first stitches configured to align the first edge of the inner skirt with the first edge of the outer skirt, the set of first stitches forming a seam that is disposed/spaced inwardly of the first edges of the inner and outer skirts. A set of second stitches configured to couple the first edge of the inner skirt with the first edge of the outer skirt, the set of second stitches being formed at the first edges of the inner and outer skirts. Wherein, the first stitches are a first type of stitch and the second stitches are a second type of stitch different from the first type of stitch.

In an aspect of the second embodiment, and in combination with any other aspects herein, the disclosure provides that the first type of stitch is one of a single running stitch and a double running stitch.

In an aspect of the second embodiment, and in combination with any other aspects herein, the disclosure provides that the second type of stitch is one of a single whipstitch and a double whipstitch.

In an aspect of the second embodiment, and in combination with any other aspects herein, the disclosure provides that the same number of second stitches are disposed within each pair of adjoining crowns.

In an aspect of the second embodiment, and in combination with any other aspects herein, the disclosure provides that there are exactly second stitches disposed within each pair of adjoining crowns.

In an aspect of the second embodiment, and in combination with any other aspects herein, the disclosure provides that the first edge of the inner skirt is an outflow edge and the first edge of the outer skirt is an outflow edge.

According to a third embodiment hereof, and in combination with any other aspects herein, the disclosure provides for a method for manufacturing a heart valve prosthesis including attaching an inner skirt to an inner portion, the inner skirt having a first edge. Attaching an outer skirt to an outer portion, the outer skirt having a first edge. Coupling the inner portion to the outer portion, wherein a plurality of adjoining crowns are defined at the points where the inner portion and the outer portion are coupled. Aligning the outflow edge of the inner skirt with the first edge of the outer skirt. Coupling the inner skirt to the outer skirt using a set of first stitches, the set of first stitches forming a seam that is configured to maintain alignment of the first edge of the inner skirt and the first edge of the outer skirt. Coupling the first edge of the inner skirt to the first edge of the outer skirt using a set of second stitches, wherein the second stitches are disposed within each pair of adjoining crowns.

In an aspect of the third embodiment, and in combination with any other aspects herein, the disclosure provides that the first stitches are a first type of stitch and the second stitches are a second type of stitch different from the first type of stitch.

In an aspect of the third embodiment, and in combination with any other aspects herein, the disclosure provides that the first type of stitch is one of a single running stitch and a double running stitch.

In an aspect of the third embodiment, and in combination with any other aspects herein, the disclosure provides that the second type of stitch is one of a single whipstitch and a double whipstitch.

In an aspect of the third embodiment, and in combination with any other aspects herein, the disclosure provides that both the set of first stitches and the set of second stitches us a thread with a diameter between 50 μm and 69 μm.

In an aspect of the third embodiment, and in combination with any other aspects herein, the disclosure provides that the same number of second stitches are disposed within each pair of adjoining crowns.

In an aspect of the third embodiment, and in combination with any other aspects herein, the disclosure provides that the first edge of the inner skirt is an outflow edge and the first edge of the outer skirt is an outflow edge.

### BRIEF DESCRIPTION OF DRAWINGS

The foregoing and other features and advantages of the invention will be apparent from the following description of embodiments thereof as illustrated in the accompanying drawings. The accompanying drawings, which are incorporated herein and form a part of the specification, further serve to explain the principles of the invention and to enable a person skilled in the art to make and use the invention. The drawings are not to scale.

FIG. 1 depicts a perspective view of a heart valve prosthesis in accordance with an aspect of this disclosure.

FIG. 2 depicts a ventricle, or bottom, outflow view of the heart valve prosthesis of FIG. 1 in accordance with an aspect of this disclosure.

FIG. 3 depicts a perspective view of an inner portion of the heart valve prosthesis of FIG. 1 in accordance with an aspect of this disclosure.

FIG. 4 depicts a perspective view of an outer portion of the heart valve prosthesis of FIG. 1 in accordance with an aspect of this disclosure.

FIG. 5 depicts a perspective view of an outflow end of the heart valve prosthesis of FIG. 1 in accordance with an aspect of this disclosure.

FIG. 6 depicts an expanded view of an area A of the heart valve prosthesis of FIG. 5 in accordance with an aspect of this disclosure.

FIG. 7 depicts a perspective view of an alternative embodiment of the heart valve prosthesis in accordance with an aspect of this disclosure.

FIG. 8 depicts an expanded view of an area A of the heart valve prosthesis of FIG. 7 in accordance with an aspect of this disclosure.

### DETAILED DESCRIPTION

Specific embodiments of the present invention are now described with reference to the figures. The terms “inflow” and “outflow”, when used in the following description refer to a native vessel, native valve, or a device to be implanted into a native vessel or native valve, such as a heart valve prosthesis, are with reference to the direction of blood flow. Thus, “inflow” refers to positions in an upstream direction with respect to the direction of blood flow and the term “outflow” refers to positions in an downstream direction with respect to the direction of blood flow.

The following detailed description is merely exemplary in nature and is not intended to limit the invention or the application and uses of the invention. Although the description of embodiments hereof is in the context of the treatment of heart valves such as the pulmonary, aortic, mitral, or tricuspid valve, the invention may also be used in other body passageways where it is deemed useful. Furthermore, there is no intention to be bound by any expressed or implied theory presented in the preceding technical field, background, brief summary or the following detailed description.

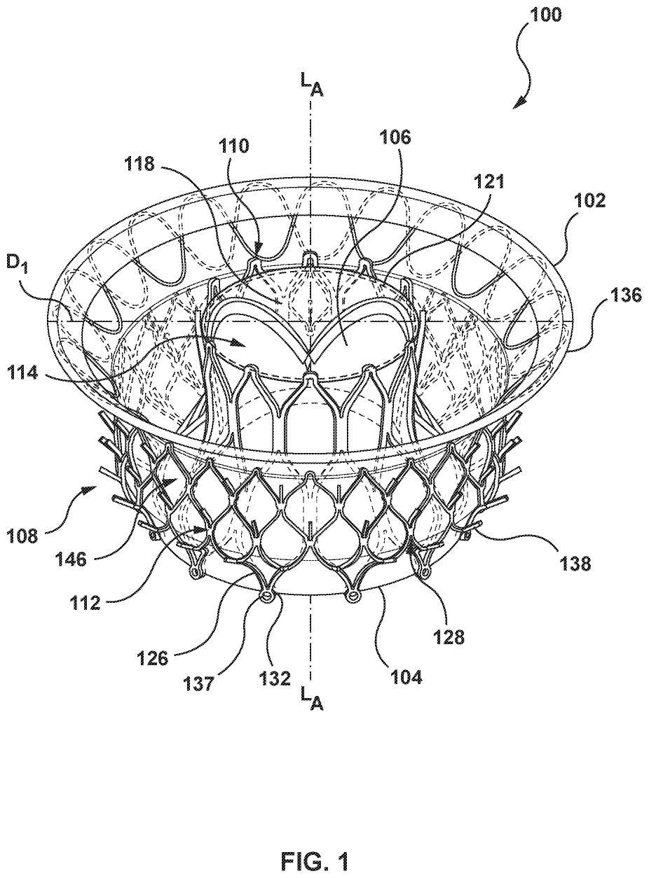

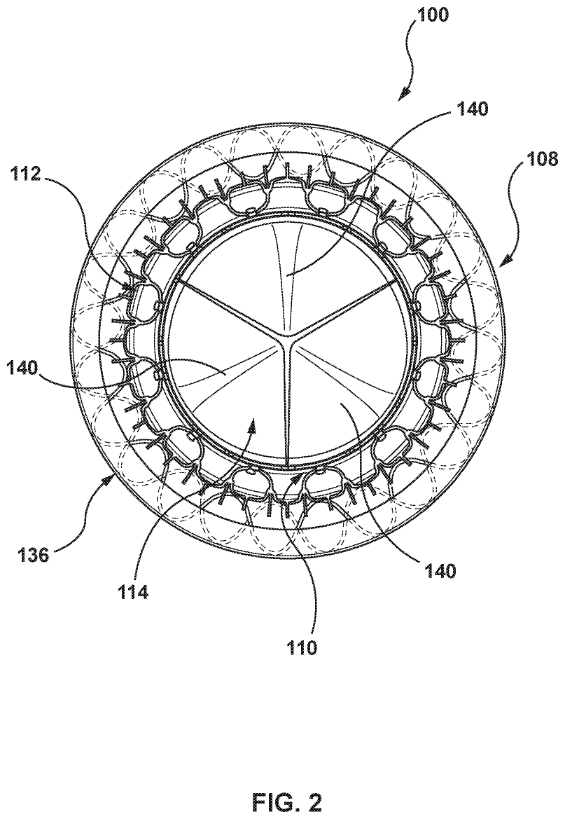

A perspective view of a transcatheter heart valve prosthesis 100 in accordance with an aspect of the disclosure is shown in FIG. 1, with FIG. 2 depicting a bottom or outflow view of the heart valve prosthesis 100. The heart valve prosthesis 100 is configured to be compressed into a reduced-diameter delivery configuration within a delivery catheter and to return to an expanded, deployed configuration when delivered/released from the delivery catheter within a native mitral valve. In some embodiments, the heart valve prosthesis 100 includes a frame 108 and a prosthetic valve component 114. The frame 108 has a stent-like structure that is configured to support the prosthetic valve component 114 and to define, along a longitudinal axis LA thereof, a blood flow lumen 106 that substantially extends from an inflow end 102 to an outflow end 104 of the heart valve prosthesis 100. In some embodiments, the frame 108 generally includes an inner portion 110 and an outer portion 112. In aspects hereof, the inner portion 110 of the frame 108 may be alternatively referred to as a valve support element, an inner frame, fixation ring, or valve housing, and/or the outer portion 112 of the frame 108 may be alternatively referred to as an anchoring element or an outer frame. The inner portion 110 is configured to hold the prosthetic valve component 114, and the outer portion 112, which surrounds the inner portion 110, is configured to secure the heart valve prosthesis 100 to the native tissue of the heart when implanted in vivo. The frame 108 may be considered to have a dual-stent structure, i.e., an inner stent and an outer stent. In some embodiments, without departing from the scope hereof, a frame 108 may be a single stent structure as to not include an outer stent structure, such as an outer portion 112.

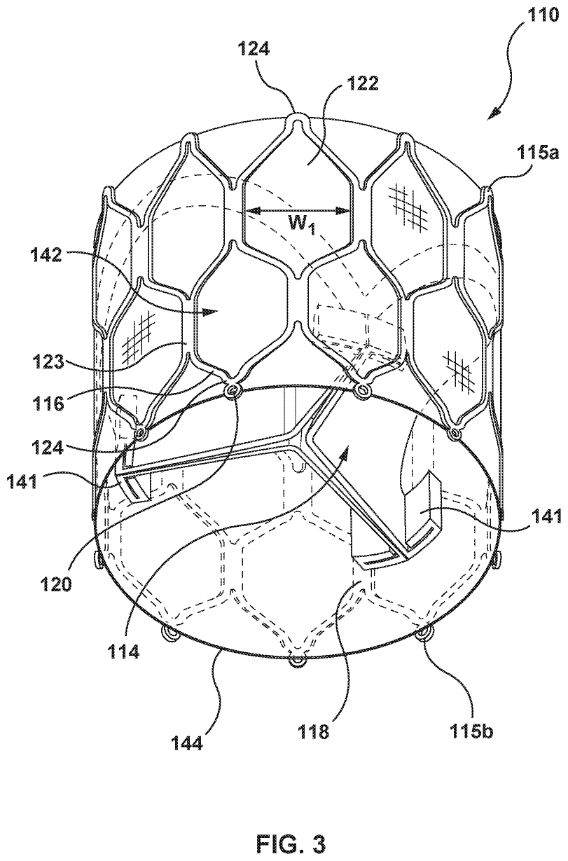

The inner portion 110 is positioned within the outer portion 112 so as to be spaced therefrom, or stated in another way, to be isolated therefrom. FIG. 3 depicts a perspective view of the inner portion 110 of the heart valve prosthesis 100 in accordance with an aspect of this disclosure and shows the interior of an outflow end 115b of the inner portion 110. The inner portion 110 generally forms a hollow cylindrical shape having a substantially constant diameter from an inflow end 115a to the outflow end 115b thereof. The stent-like structure of the inner portion 110 defines a plurality of open cells 122 arranged in a honeycomb pattern. Further, the inner portion 110 has a plurality of crowns 124 positioned at the inflow end 115a and the outflow end 115b and, in some embodiments, the crowns 124 at the outflow end 115b may have an aperture 120 that allows for the inner portion 110 to be coupled to the outer portion 112 as described in detail below. In an aspect hereof, an outflow end and an inflow end may be switched for an inner portion 110, and/or alternatively referred to as a first end or a second end, without departing from the scope hereof.

Each cell 122 of the inner portion 110 is defined by a series of struts 116, with one end of each cell 122 being defined by an endmost crown 124 and the other end of each cell 122 being defined by a node 123, with the node 123 being a thicker strut segment formed between adjacent cells 122. The node 123 may be defined as a connection point of various struts 116. A width W of each cell 122 is also, a width W beneath each crown 124, as shown in FIG. 3. The width W is determined by the overall geometry of the frame 108. The inner portion 110 is shown in the embodiment of FIG. 3 as having two rows of cells 122, however, this is merely exemplary and additional rows of cells 122 or patterns of cells 122 is envisioned without departing from the scope of the present disclosure.

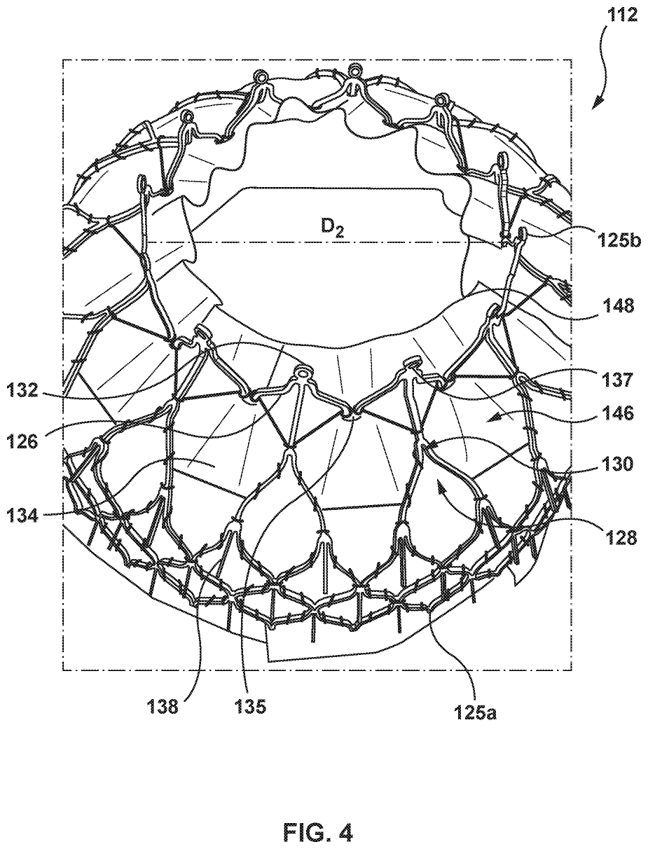

The outer portion 112 is configured to secure the heart valve prosthesis 100 to the native valve and the surrounding subannular tissue, such as the inward facing-surface of the leaflets. A perspective view of the outer portion 112, from its outflow end 125b, is shown in FIG. 4. The outer portion 112 is positioned around the inner portion 110 and defines an inflow end 125a having a first diameter D1 and an outflow end 125b having a second diameter D2 that is smaller than the first diameter D1. A transition portion 128 is positioned between the inflow end 125a and the outflow end 125b of the outer portion 112. As shown in FIG. 4, the transition portion 128 reduces in diameter between the inflow end 125a and the outflow end 125b, acting as a taper between the first diameter D1 and the second, smaller diameter D2. In an aspect hereof, an outflow end and an inflow end may be switched for an outer portion 112, and/or alternatively referred to as a first end or a second end, without departing from the scope hereof.

At least a portion of the outer surface of the outer portion 112, when the heart valve prosthesis 100 is in an expanded state, is configured to be disposed against the native tissue of the heart for securing the outer portion 112 and, concurrently, the heart valve prosthesis 100. Further, the outer portion 112 is mechanically isolated from the inner portion 110. In more detail, the outer portion 112 may deform upon implantation within a native mitral valve annulus, and/or expand and contract in response to movement of the native tissue, while remaining spaced from the inner portion 110, which thereby permits the inner portion 110 to remain relatively still and undeformed. The inner portion 110 is, therefore, isolated from external forces, allowing for the prosthetic valve component 114 to more efficiently replicate the function of the native mitral valve. In addition, the outer portion 112 may further include a plurality of prongs 138 that extend radially from the outer surface of the outer portion 112 and are configured to engage with the native tissue, further fixating the outer portion 112 to the tissue.

To further explain the stent-like or lattice structure of the outer portion 112, a plurality of cells 134 are defined by a plurality of struts 126 of the outer portion 112. At the outflow end 125b of the outer portion 112 a plurality of first crowns 132 and a plurality of second crowns 135 are formed by respective pairs of opposing struts 126, wherein each first crown 132 is circumferentially spaced from an adjacent crown 132 by a second crown 135. The plurality of second crowns 135 are spaced inwardly of the plurality of first crowns 132 with struts 126 extending therebetween so as to form a substantially wavy or zig-zag patterned ring at the outflow end 125b of the outer portion 112 that is sized and configured to substantially correspond to the pattern of crowns 124, nodes 123 and struts 116 at the outflow end 115b of the inner portion 110. Further, the first crowns 132 may be coupled to a plurality of Y-shaped struts 130 that make-up the transition portion 128 and provide a transition from the first diameter D1 to the second diameter D2 by bending inward towards the longitudinal axis LA. In alternative embodiments, it is envisioned that the plurality of Y-Bars 130 may make up a portion or the entirety of the transition portion 128. In some embodiments, there is an equivalent number of second crowns 135 as there are first crowns 132. Further, each first crown 132 of the outer portion 112 has an aperture 137 that allows for the outer portion 112 to be coupled or connected to a corresponding crown 124 of the inner portion 110.

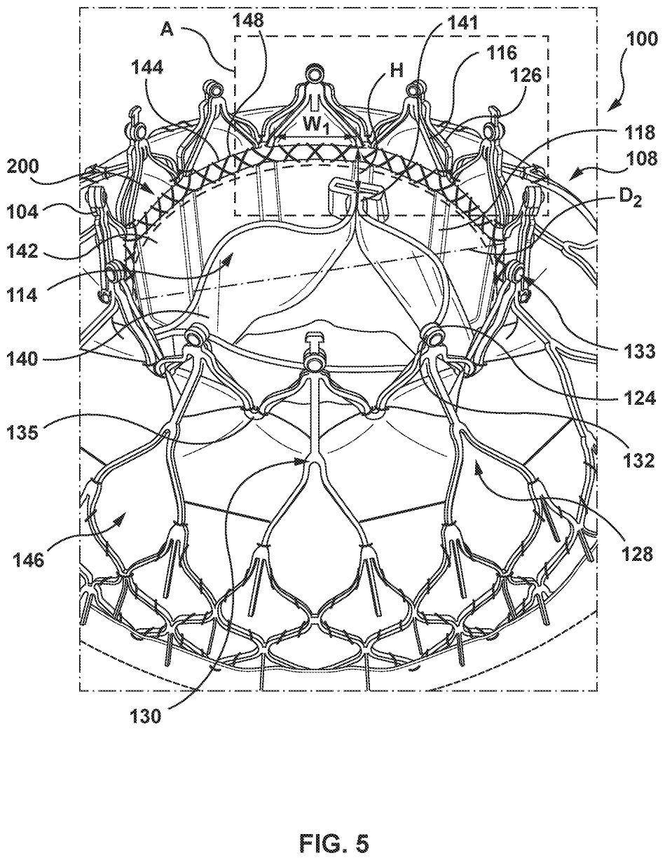

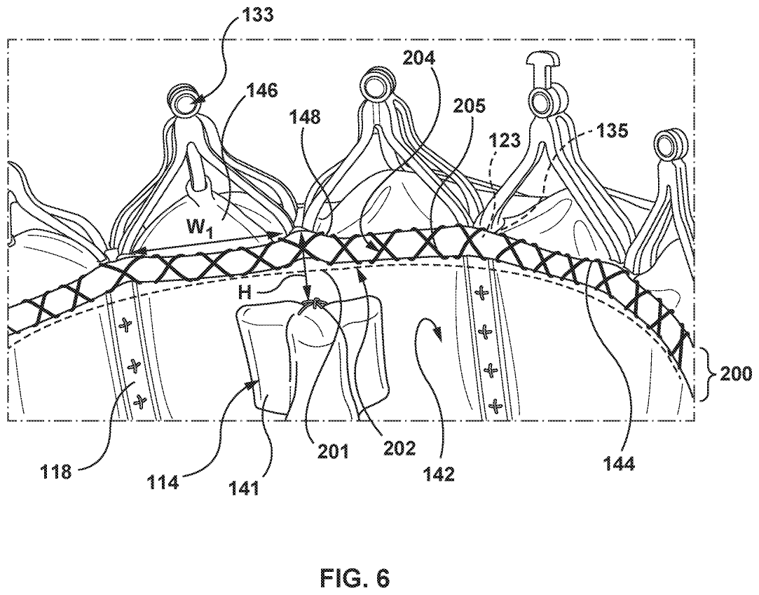

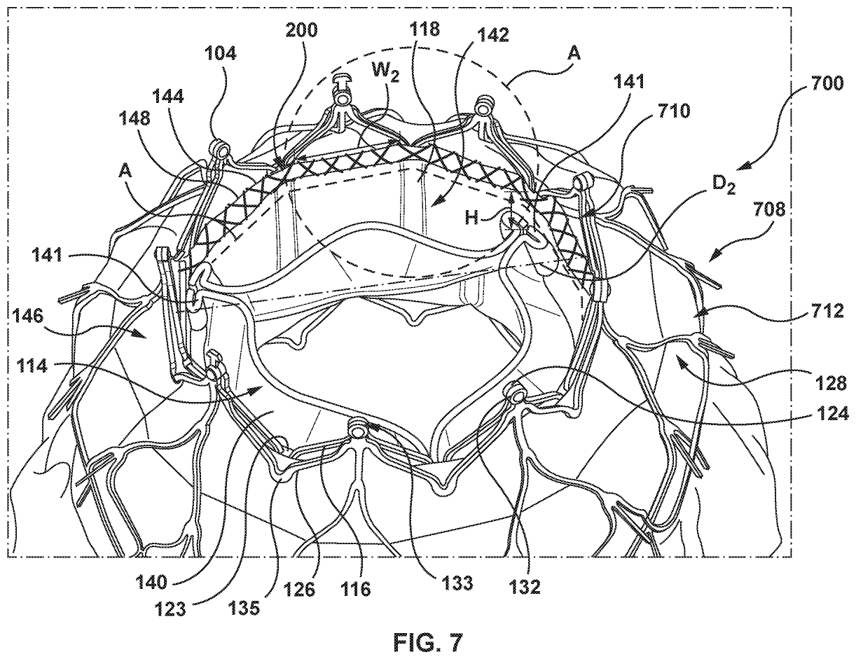

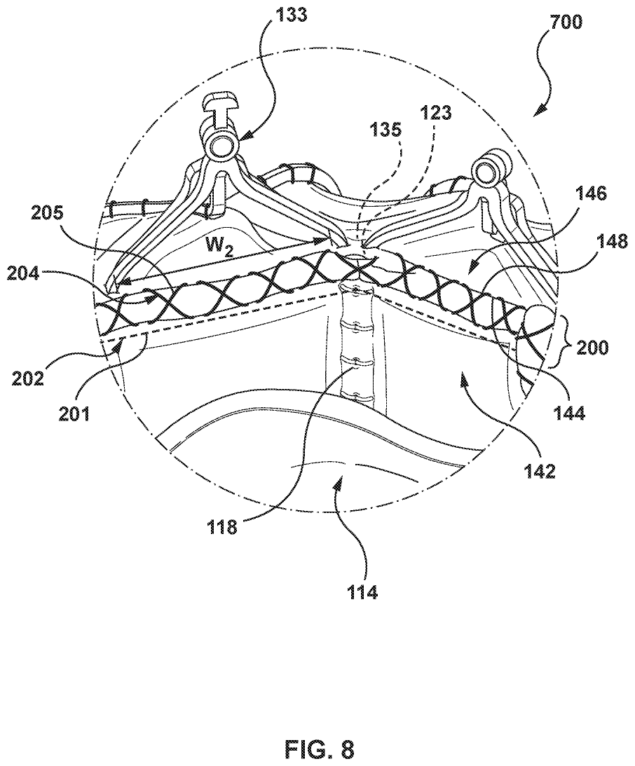

As described above, the plurality of crowns 124 at the outflow end 115b of the inner portion 110 and the plurality of first crowns 132 at the outflow end 125b of the outer portion 112 have apertures 120, 137 that align with one and other to permit the coupling together of the inner portion 110 and the outer portion 112. When one of the crowns 124 of the inner portion 110 is coupled to one of the first crowns 132 of the outer portion 112, a pair of adjoining crowns 133 is formed and, depending on the configuration of the inner portion 110 and the outer portion 112, the number of pairs of adjoining crowns 133 may vary. Further, the pairs of adjoining crowns 133 may be joined by rivets, welding, or other methods known in the art. The pairs of adjoining crowns 133 is shown in FIGS. 5 and 6, with FIG. 5 being a perspective view of the heart valve prosthesis 100 from the outflow end 104 and FIG. 6 being an enlarged view of an area A of FIG. 5. The heart valve prosthesis 100 is shown in FIGS. 1-6 as having twelve pairs of adjoining crowns 133, however, this is merely an exemplary heart valve prosthesis 100 and configurations of the heart valve prosthesis 100 are envisioned that may include other numbers of pairs of adjoining crowns 133. For example, FIGS. 7 and 8 show a heart valve prosthesis 700 having a frame 708 with an inner portion 710 and outer portion 712 and nine pairs of adjoining crowns 133, with FIG. 7 being a perspective view of the heart valve prosthesis 700 from an outflow end 104 thereof and FIG. 8 being an enlarged view of an area A of FIG. 7. The embodiment of FIGS. 7 and 8 shares the same features and functions as previously described for the embodiment of FIGS. 1-6 unless otherwise noted.

Each of the embodiments of FIGS. 5 and 7 have the same second or outflow diameter D2 at an outflow end 104 of the heart valve prosthesis 100, 700 shown therein. As noted above, the heart valve prosthesis 100 has a larger number of pairs of adjoining crowns 133 than the heart valve prosthesis 700 and consequently a width W1 beneath a pair of adjoining crowns 133 of the heart valve prosthesis 100, as shown in FIGS. 5 and 6, is less than a width W2 beneath a pair of adjoining crowns 133 of the heart valve prosthesis 700, as shown in FIGS. 7 and 8. Width W1 of the heart valve prosthesis 100 is less than width W2 of the heart valve prosthesis 700 because its respective inner portion 110 has a greater number of cells 122 than a number of cells 122 of the inner portion 710 of the heart valve prosthesis 700. In other words, for a given outflow diameter, a crown or cell width will increase as a number of pairs of adjoining crowns decreases, and a crown or cell width will decrease as a number of pairs of adjoining crowns increases. Therefore, it follows that a change in an outflow or second diameter D2 may also change a crown or cell width W. For example, if the outflow or second diameter D2 is increased and the number of pairs of adjoining crowns 133 stays constant, a crown or cell width W must increase.

In accordance with aspects hereof, the inner portion 110 and the outer portion 112 of the frame 108 of the heart valve prothesis 100 may be made from any number of suitable biocompatible materials, e.g., stainless steel, nickel titanium alloys such as Nitinol™, cobalt chromium alloys such as MP35N, other alloys such as ELGILOY® (Elgin, Ill.), various polymers, pyrolytic carbon, silicone, polytetrafluoroethylene (PTFE), or any number of other materials or combination of materials. A suitable biocompatible material would be selected to provide the heart valve prosthesis 100 to be configured to be compressed into a reduced diameter configuration for transcatheter delivery to a native valve, whereby release from a delivery catheter allows the prosthesis 100 to self-expand, returning to an expanded, deployed configuration. In some embodiments, the self-expansion is accomplished through the use of a shape-memory material such as Nitinol™. The heart valve prosthesis 100 may be processed to have a default or “set” shape that coincides with the deployed configuration. Therefore, once the compressed heart valve prosthesis 100 is delivered and released, the prosthesis 100 will return to the default or “set” deployed configuration.

The prosthetic valve component 114 of the heart valve prosthesis 100 is capable of regulating flow therethrough via valve leaflets 140. FIGS. 1-3 and 5-8 illustrate an exemplary prosthetic valve component 114 having three leaflets 140, although a bicuspid leaflet configuration may alternatively be used in embodiments hereof When deployed in situ, the prosthetic valve component 114 in a closed state is configured to block blood flow in one direction to regulate blood flow through the blood flow lumen 106 of the inner portion 110. The valve leaflets 140 are disposed to coapt within the inner portion 110 and are secured to the inner surface 118 of the inner portion 110, such that the valve leaflets 140 open during diastole. The leaflets 140 are attached along their bases to the inner portion 110, for example, using sutures or a suitable biocompatible adhesive. Adjoining pairs of leaflets 140 are attached to one another at their lateral ends to form leaflet commissures 141. The orientation of the leaflets 140 within the inner portion 110 depends upon which end of the heart valve prosthesis 100 is the inflow end 102 and which end of the heart valve prosthesis 100 is the outflow end 104, thereby ensuring one-way flow of blood through the heart valve prosthesis 100. As shown in FIG. 5, the prosthetic valve component 114 is operably coupled to the inner portion 110 at a distance H inwardly from the nodes 123. The distance H is the measured axial displacement between the commissures 141 of the prosthetic valve component 114 and the nodes 123 of the inner portion 110. By displacing the prosthetic valve component 106 the distance H the prosthetic valve component 106 is encompassed inside the inner skirt 118, thereby protecting the prosthetic valve component 106 from being pinched, cut, or otherwise damaged. Further, the distance Hi can be optimized to the configuration and design of the frame 102 as to avoid or reduce the risk of damage to the prosthetic valve component 106.

Further, the leaflets 140 may be formed of various flexible materials including, but not limited to, natural pericardial material such as tissue from bovine, equine or porcine origins, or synthetic materials such as polytetrafluoroethylene (PTFE), DACRON® polyester, pyrolytic carbon, or other biocompatible materials. With certain prosthetic leaflet materials, it may be desirable to coat one or both sides of the replacement valve leaflet with a material that will prevent or minimize overgrowth. It is further desirable that the prosthetic leaflet material is durable and not subject failure due to stretching, deforming, or fatigue.

The heart valve prosthesis 100 may further include a brim or preshaped wire element 136 that extends outwardly from the inflow end 125a of the outer portion 112. The brim 136 includes overlapping, 180 degree out of phase sinusoidal wire forms that are attached and hinged to the outer portion 112 by a suitable biocompatible low-profile fabric used in bioprosthetic implants namely endovascular grafts, valves or left atrial appendage devices to promote bio-integration, such as woven polyethylene terephthalate (PET) fabric. The brim element 136 may act as an atrial retainer, if present, and to serve such a function the brim element 136 may be configured to engage tissue above a native annulus, such as a supra-annular surface or some other tissue in the left atrium, to thereby inhibit downstream migration of the heart valve prosthesis 100 as well as mitigate any leakage through any gaps between native tissue and the brim, for e.g., during atrial systole.

An inner skirt 142 is disposed within and is coupled to the inner portion 110, and, more particularly, is coupled as to line an inner surface 118 of the inner portion 110, or at least a substantial portion thereof. In an aspect hereof, the inner skirt 142 extends from the inflow end 115a of the inner portion 110 to the outflow end 115b of the inner portion 110, with an outflow edge 144 of the inner skirt 142 being defined at the outflow end 115b of the inner portion 110. When the heart valve prothesis 100 is implanted within a native mitral valve, the inner skirt 142 is configured to limit the amount of unintentional blood leakage, otherwise known as regurgitation, between a left atrium and a left ventricle. In further detail, the inner skirt 142 covers the inner surface 118 of the inner portion 110, as to only allow blood to flow from the atrium to the ventricle when the prosthetic valve component 114 is in the open state. The inner skirt 142 may take the form of a single piece or multiple pieces of material that is wrapped within the inner surface 118 as to create a cylindrical body that is flush with the inner surface 118. The inner skirt 142 is then affixed to the inner portion 110 using sutures or adhesive. In order to inhibit blood flow, the inner skirt 142 is further configured to substantially cover the cells 122 of the inner portion 110.

Similarly, an outer skirt 146 is disposed within and is coupled to the outer portion 112. In more detail, the outer skirt 146 extends from the inflow end 125a of the outer portion 112 to the outflow end 125b of the outer portion 112. As discussed previously, the outer portion 112 has the first diameter D1 at the inflow end 125a that is larger than the second diameter D2 at the outflow end 125b, therefore, the outer skirt 146 is configured to match the tapered profile of the transition portion 128. When the heart valve prothesis 100 is implanted within a native mitral valve, the outer skirt 146 substantially covers the inner surface of the outer portion 112 so as to limit unintentional blood flow from the left atrium to the left ventricle. The outer skirt 146 may take the form of a single piece or multiple pieces of material that is wrapped within the inner surface as to create a shape that is flush with the outer portion 112. The outer skirt 146 is then affixed to the outer portion 112 using sutures or adhesive. In order to inhibit blood flow, the outer skirt 146 is further configured to substantially cover the cells 134 of the outer portion 112. By displacing the prosthetic valve component 106 the distance Hi, the prosthetic valve component 106 is encompassed inside the inner skirt 118, thereby protecting the prosthetic valve component 106 from being pinched, cut, or otherwise damaged. Further, the distance Hi can be optimized to the configuration and design of the frame 102 as to avoid or reduce the risk of damage to the prosthetic valve component 106.

The inner skirt 142 is coupled to the inner portion 110 and the outer skirt 146 is coupled to the outer portion 112 prior to the inner portion 110 and the outer portion 112 being coupled to one another. Therefore, when the inner portion 110 is coupled to the outer portion 112, the outer skirt 146 is positioned outside of the inner portion 110 and, subsequently, is pinched between the nodes 123 of the inner portion 110 and the second crowns 135 of the outer portion 112.

In aspects hereof, the inner skirt 142 and the outer skirt 146 may be formed of a low-porosity woven fabric, such as polyester, Dacron fabric, or PTFE. In further aspect, the inner and outer skirts 142, 146 may be a knit polyester, such as a polyester or PTFE knit, which can be used when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side. These and other appropriate cardiovascular fabrics are commercially available from Bard Peripheral Vascular, Inc. of Tempe, Ariz., for example. In alternative aspects hereof, the inner and outer skirts 142, 146 may be formed of a natural or biological material such as pericardium or another membranous tissue such as intestinal submucosa. Further, it is envisioned that the inner skirt 142 and the outer skirt 146 may be made of the same or different materials, for example, the inner skirt 142 may be made from a PTFE knit, while the outer skirt 146 is made of a woven polyester. It is further envisioned, that in some embodiments, it may be beneficial to have skirts of varying thicknesses, such as, an inner skirt 142 that is thicker than an outer skirt 146.

In order to further limit any unintentional blood leakage, the inner skirt 142 and the outer skirt 146 are coupled together using a skirt seal 200. The skirt seal 200 is shown in FIGS. 5, 6, 7, and 8. In further detail, the inner skirt 142 has an outflow edge 144, shown in FIG. 3, and the outer skirt 146 has an outflow edge 148, as shown in FIG. 4. Both outflow edges 144, 148 are free edges when the inner portion 110 and the outer portion 112 are coupled to one another, and, therefore, the outflow edges 144,148 of the inner and outer skirts 142, 146 are coupled together to limit or prevent regurgitation. The skirt seal 200 extends about an inner circumference 121 of the inner portion 110 and is disposed axially at or inwardly from the free edges of the outflow edges 144, 148 of the inner and outer skirts 142, 146.

In an embodiment, the skirt seal 200 is formed by aligning the outflow edge 144 of the inner skirt 142 with the outflow edge 148 of the outer skirt 146 and creating a seam 201, using a set of first stitches 202, that is disposed inwardly of the outflow edges 144, 148. In some embodiments, once the seam 201 is created, the skirt seal 200 includes a set of second stitches 204 being made at or over the outflow edges 144,148 of the inner and outer skirts 142, 146, with the set of second stitches 204 being formed to further couple and seal the inner skirt 142 to the outer skirt 146. The skirt seal 200 may be disposed at the inner surface 118 of the inner portion 110 and run parallel relative to the nodes 123 of the inner portion 110. Further, the skirt seal 200 may be positioned at the location of the nodes 123, however, it is envisioned that the skirt seal 200 may also be positioned inward or outward relative to the nodes 123. For example, the skirt seal 200 may be positioned inward relative to the nodes 123 and outward relative to the prosthetic valve component 114.

The seam 201 of the set of first stitches 202, shown in FIGS. 6 and 8, is configured to hold or secure the alignment of the outflow edges 144, 148 of the inner and outer skirts 142, 146. In an aspect hereof, each of the first stitches 202 may be a first type of stitch known as a running or straight stitch, which is a small even stitch that runs in and out through a material or fabric. The set of first stitches 202 may be a series of running, or straight, stitches, which is a series of small even stitches that run in and out through the material without overlapping. In an aspect, the seam 201 of the set of first stitches 202, i.e., running stitches 202, is positioned inward from the outflow edges 144, 148, to encircle the perimeter of both the inner skirt 142 and the outer skirt 146. In an aspect hereof, the seam 201 may be spaced inward between 0.5 mm and 2 mm from the outflow edges 144, 148 and set of first stitches 202 of the seam 201 may be disposed in a straight line that is parallel with the outflow edges 144, 148 of the inner and outer skirts 142, 146. In an aspect, each running or straight first stitch 202 of the seam 201 may measure between 0.5 mm and 2.5 mm in length to ensure that the seam 201 is strong enough to assure the alignment of the outflow edges 144, 148. In an aspect, the set of first stitches 202 that comprise the seam 201 may be comprised of a first type of stitch referred to as a “double” running stitch. In an aspect, double running stitches are made to form a seam 201 by initially creating a straight line of first running stitches with spaces between adjacent first running stitches, and secondly going back over the straight line of first running stitches, in a reverse direction, and filling in the spaces between adjacent first running stitches with a straight line of second running stitches. Though FIGS. 6 and 8 both display a seam 201 in which the set of first stitches 202 is a series of “double” running stitches, it is envisioned that a seam of single running stitches, or other methods or stitching techniques known in the art to align the outflow edges 144,148 may be used.

The set of second stitches 204 of the skirt seal 200 are shown in both FIGS. 6 and 8 and are used to further couple and seal the outflow edges 144, 148, after the outflow edges 144,148 have been aligned by the seam 201 created by the set of first stitches 202. In an aspect hereof, each stitch of the set of second stitches 204 may be a second type of stitch known as a whipstitch, which is an overcast stitch made over a fabric or material edge. The set of second stitches 204 may be a series of whipstitches that are made over the outflow edges 144, 148 of the inner and outer skirts 142, 146, around an entire perimeter thereof, to couple and seal them together.

In an aspect, the set of second stitches 204 may be comprised of a second type of stitch referred to as a double whipstitch. In an aspect, the set of double whipstitches 204 may be formed by firstly creating a line of first whipstitches spaced around the perimeter of the aligned outflow edges 144, 148, with each first whipstitch being sewn at a 45-degree angle with reference to the aligned edges. Secondly, going back over the line of first whipstitches in a reverse direction, creating a line of second whipstitches spaced around the perimeter of the aligned outflow edges 144, 148, with each second whipstitch being sewn over a corresponding first whipstitch at a 135-degree angle with reference to the aligned edges. As such, the “double” whipstitches resemble a plurality of X-shaped double whipstitches 205 spaced around the outflow edges 144, 148 of the inner and outer skirts 142, 146. The overlapping nature of the set of second stitches 204 being formed by double whipstitches 205 creates a stronger and more robust connection between the two pieces of material, i.e., the inner skirt and outer skirt, and further limits any unintentional blood flow at the location of the skirt seal 200. Though FIGS. 6 and 8 both display a series of double whipstitches 205, it is envisioned that either a series of single whipstitches or other methods or stitching techniques known in the art to seal the outflow edges 144, 148 may be used.

With reference to FIGS. 6 and 8, each of the double whipstitches 205 is positioned under a pair of adjoining crowns 133 within the width W1, W2 and, in some embodiments, the same number of double whipstitches 205 are positioned under each pair of adjoining crowns 133. To evenly secure the inner skirt 142 to the outer skirt 146, each double whipstitch 205 may be placed at an equal distance, for example between 1.25 mm to 1.5 mm, from an adjacent double whipstitch 205, and, therefore, the number of double whipstitches 205 positioned beneath each pair of adjoining crowns 133 will depend on the overall cell width W of the inner portion 110. For example, as shown in FIG. 6, three double whipstitches 205 are placed under each of the pair of adjoining crowns 133 within the width W1, while in FIG. 8, four double whipstitches 205 are placed under each of the pair of adjoining crowns 133 within the greater width W2. To explain further, it is envisioned that the number of double whipstitches 205 is determined by the size of the cell width W, for example, in FIG. 6, the demonstrated frame 108 has a total of twelve pairs of adjoining crowns 133 resulting in a smaller cell width W1 when compared to a cell width W2 of the nine pairs of adjoining crowns 133 of FIG. 8. Therefore, a larger number of double whipstitches 205 will be necessary to properly and evenly secure the inner skirt edge 144 to the outer skirt edge 148 as the cell width W increases. The number of double whipstitches 205 that are positioned under each adjoining crown 133 in FIGS. 6 and 8 are merely exemplary and it is envisioned that a larger or smaller number of double whipstitches 205 may be positioned under each adjoining crown 133 as may be suitable for a given application to provide proper sealing.

Further, each double whipstitches 205 may be used to secure the inner skirt 142 and the outer skirt 146 to the inner portion 110 and the outer portion 112. Referring to FIG. 7 for example, five double whipstitches 205 are shown, wherein three are disposed inward from the pair of adjoining crowns 133, and the remaining two double whipstitches 205 are positioned at anode 123 of the inner portion 110 and a second crown 135 of the outer portion 112. The double whipstitches 205 positioned at the nodes 123 and second crowns 135 are configured to secure both the inner skirt 142 and the outer skirt 146 to the inner portion 110.

In some embodiments, sutures are used to stitch the inner skirt 142 and the outer skirt 146 together. The sutures may be a natural or biological material such as pericardium or another membranous tissue such as intestinal submucosa. Alternatively, the sutures may be a low-porosity woven fabric, such as polyester, Dacron fabric, or PTFE. Further, the diameter of the suture may be configured to either increase the strength of the stitching or to reduce the packing volume of the heart valve prosthesis 100. For example, in some embodiments, using a suture having a diameter between 50 μm and 69 μm will reduce packing size of the heart valve prosthesis 100 while maintaining the strength to sufficiently couple the inner skirt 142 and the outer skirt 146 together to form the skirt seal 200. In some embodiments, DYNEEMA 7-0 or 5-0 sutures may be used. However, other sized sutures 206 having diameters ranging between 20 μm and 150 μm are envisioned.

In order to create the skirt seal 200, the inner skirt 142 is first coupled to the inner portion 110 and the outer skirt 146 is coupled to the outer portion 112, wherein the skirts 142, 146 are coupled to their respective portions 110, 112 using sutures, adhesives, or other methods known in the art. The inner portion 110 and the outer portion 112 are then coupled together, in some embodiments, this coupling is done using a series of rivets positioned at the apertures 120, 137 of the inner and outer portions 110, 112. Once the inner and outer portions 110, 112 are coupled, the outflow edge 144 of the inner skirt 142 is then aligned with the outflow edge 148 of the outer skirt 146. The set of first stitches 202 is then used to create a seam 201, wherein the seam 201 is positioned inwardly relative to the aligned outflow edges 144, 148. Further, the first stitches 202 may be a first type of stitch such as a single or double running stitch as described above. Once the seam 201 is created, the outflow edges 144, 148 are then coupled to one another using the set of second stitches 204, the second stitches 204 may be a second type of stitch such as a series of single whipstitch or a series of double whipstitches. Further, in an aspect hereof a uniform number of double whipstitches 205 are disposed beneath each pair of adjoining crowns 133 as to ensure that the skirt seal 200 limits the amount of unintentional blood flow between the atrium to the ventricle.

In embodiments hereof, the benefit of using a skirt seal 200 is not limited for use in a heart valve prosthesis 100 having the particular features disclosed above but instead may be adapted for use in valve prosthesis designed for other uses as would be recognized by one of ordinary skill in the art upon considering this disclosure. For instance in certain applications, a skirt seal 200 may be made at an inflow end of a prosthesis as may be appropriate.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A transcatheter heart valve prosthesis comprising:
a frame including
an inner portion configured to support a prosthetic valve component, and
an outer portion coupled to the inner portion, the outer portion being sized to surround the inner portion and configured to anchor the prosthesis;
an inner skirt disposed within and coupled to the inner portion, the inner skirt having a first edge;
an outer skirt disposed within and coupled to the outer portion, the outer skirt having a first edge; and
a skirt seal configured to provide a seal between the first edges of the inner skirt and the outer skirt, the skirt seal including
a set of first stitches configured to align the first edge of the inner skirt with the outflow edge of the outer skirt, the set of first stitches forming a seam that is disposed/spaced inwardly of the first edges of the inner and outer skirts, and
a set of second stitches configured to couple the first edge of the inner skirt with the first edge of the outer skirt, the set of second stitches being formed at the first edges of the inner and outer skirts,
wherein the first stitches are a first type of stitch, and the second stitches are a second type of stitch different from the first type of stitch.

2. The prosthesis of claim 1, wherein the inner portion has a stent-like structure with a second end and a first end, the stent-like structure including a plurality of crowns defining apertures at the outflow end.

3. The prosthesis of claim 2, wherein the outer portion has a stent-like structure with a second end and a first end, the stent-like structure including a plurality of crowns defining apertures at the first end.

4. The prosthesis of claim 3, wherein the plurality of the crowns at the first end of the outer portion are coupled to the plurality of crowns at the first end of the inner portion so as to form a plurality of pairs of adjoining crowns.

5. The prosthesis of claim 4, wherein the first edge of the inner skirt is an outflow edge and the first edge of the outer skirt is an outflow edge.

6. The prosthesis of claim 5, wherein the same number of second stitches are disposed within each pair of adjoining crowns.

7. The prosthesis of claim 6, wherein exactly three second stitches are disposed within each pair of adjoining crowns.

8. The prosthesis of claim 1, wherein the first type of stitch is a double running stitch.

9. The prosthesis of claim 1, wherein the first type of stitch is a single running stitch.

10. The prosthesis of claim 1, wherein the second type of stitch is a double whipstitch.

11. The prosthesis of claim 1, wherein the set of first stitches and the set of second stitches are formed by a suture having a diameter of between 50 μm and 69 μm.

12. A transcatheter heart valve prosthesis comprising:
a frame including:
an inner portion having a plurality of crowns at a first end thereof, and an outer portion having a plurality of crowns at a first end thereof, the plurality of crowns of the outer portion being coupled to the plurality of crowns of the inner portion so as to form a plurality of pairs of adjoining crowns;
a prosthetic heart valve coupled to the inner portion;
an inner skirt coupled to the inner portion, the inner skirt having a first edge;
an outer skirt coupled to the outer portion and having a first edge;
a skirt seal configured to provide a seal between the first edges of the inner skirt and the outer skirt, the skirt seal including
a set of first stitches configured to align the first edge of the inner skirt with the first edge of the outer skirt, the set of first stitches forming a seam that is disposed/spaced inwardly of the first edges of the inner and outer skirts, and
a set of second stitches configured to couple the first edge of the inner skirt with the first edge of the outer skirt, the set of second stitches being formed at the first edges of the inner and outer skirts;
wherein the first stitches are a first type of stitch, and the second stitches are a second type of stitch different from the first type of stitch.

13. The prosthesis of claim 12, wherein the first type of stitch is one of a single running stitch and a double running stitch.

14. The prosthesis of claim 12, wherein the second type of stitch is one of a single whipstitch or a double whipstitch.

15. The prosthesis of claim 12, wherein the same number of second stitches are disposed within each pair of adjoining crowns.

16. The prosthesis of claim 15, wherein there are exactly three second stitches disposed within each pair of adjoining crowns.

17. The prosthesis of claim 12, wherein the first edge of the inner skirt is an outflow edge and the first edge of the outer skirt is an outflow edge.

18. A method for manufacturing a heart valve prosthesis comprising:
attaching an inner skirt to an inner portion, the inner skirt having a first edge;
attaching an outer skirt to an outer portion, the outer skirt having a first edge;
coupling the inner portion to the outer portion, wherein a plurality of adjoining crowns are defined at the points where the inner portion and the outer portion are coupled;
aligning the first edge of the inner skirt with the first edge of the outer skirt;
coupling the inner skirt to the outer skirt using a set of first stitches, the set of first stitches forming a seam that is configured to maintain alignment of the first edge of the inner skirt and the first edge of the outer skirt;
coupling the first edge of the inner skirt to the first edge of the outer skirt using a set of second stitches, wherein the second stitches are disposed within each pair of adjoining crowns.

19. The method of claim 18, wherein the first stitches are a first type of stitch and the second stitches are a second type of stitch different from the first type of stitch.

20. The method of claim 19, wherein at least the first type of stitch or the second type of stich is one of a single running stitch and a double running stitch.
